(19) United States
(12) Patent Application Publication
(10) Pub. No.: US 2008/0241892 A1
(43) Pub. Date: Oct. 2, 2008

(54) Modified surfaces for immobilization of active molecules

(75) Inventors: Daniel Bernardo Roitman, Menlo Park, CA (US); Geoff Otto, Santa Clara, CA (US); Ronald L. Cicero, Palo Alto, CA (US); Nelson R. Holcomb, Palo Alto, CA (US)

Correspondence Address: QUINE INTELLECTUAL PROPERTY LAW GROUP, P.C., P O BOX 458, ALAMEDA, CA, 94501, US

(73) Assignee: Pacific Biosciences of California, Inc., Menlo Park, CA (US)

(21) Appl. No.: 12/079,922

(22) Filed: Mar. 27, 2008

Related U.S. Application Data
(60) Provisional application No. 60/921,085, filed on Mar. 29, 2007.

(51) Int. Cl. C12P 19/34; B32B 15/08; C40B 50/14; C40B 50/06; B32B 27/00; C12P 1/00; C07H 21/00; C12N 11/14

(52) U.S. Cl. 435/91.2; 428/457; 506/30; 506/26; 428/411.1; 435/41; 536/23.1; 435/174; 428/702

(57) ABSTRACT

Modified surfaces, substrates, and methods of producing and using such substrates and surfaces are provided. The substrates and surfaces provide either non-reactive surfaces or low density reactive groups, preferably on an otherwise non-reactive surface, for use in different applications including single molecule analyses.

### CROSS-REFERENCE TO RELATED APPLICATIONS

This application is a non-provisional utility patent application claiming priority to and benefit of the following prior provisional patent application: U.S. Ser. No. 60/921,085, filed Mar. 29, 2007, entitled “MODIFIED SURFACES FOR IMMOBILIZATION OF ACTIVE MOLECULES” by Daniel Bernardo Roitman et al., which is incorporated herein by reference in its entirety for all purposes.

### FIELD OF THE INVENTION

The present invention relates to methods of producing modified surfaces and substrates. The substrates and surfaces provide either non-reactive surfaces or low density reactive groups, preferably on an otherwise non-reactive surface, for use in applications such as single molecule analyses.

### BACKGROUND OF THE INVENTION

Understanding, or lack thereof, as to the characteristics of a surface and its interactions with its environment has been at the center of monumental discoveries, as well as monumental failures, in materials science. This issue permeates virtually every technological endeavor, whether it is in the field of engineering, chemistry, or biology, whether it is focused on nanomaterials technology, extraterrestrial exploration, semiconductor technology, biotechnology manufacturing, or pharmaceutical administration and delivery. While understanding the bulk properties of a material presents one problem, the point at which that material ceases, where one must understand and/or deal with the properties of the surface of that material and how that surface will interact with its environment, is something altogether different.

The present invention is directed at materials and/or their surfaces that are selected and/or configured to meet a variety of different needs, including, inter alia, a capacity and ability to selectively bind to desired molecules while preventing excessive binding of undesired molecules. Other advantageous characteristics will be apparent upon reading the following disclosure.

### SUMMARY OF THE INVENTION

The present invention is generally directed to substrates bearing modified surfaces that are useful in a variety of different, useful applications, as well as methods of producing such substrates and uses and applications of these substrates. In particular, the substrates of the invention possess surfaces with a selected density of reactive groups disposed on that surface, and preferably, a selected low density of such reactive groups. Optionally, the surfaces are non-reactive.

A first general class of embodiments provides methods of preparing a modified surface. In the methods, a surface to be modified is provided. At least three different monomers are copolymerized to form a polymer, wherein the at least three monomers comprise a first monomer comprising an alkyl phosphonate or alkyl phosphate group, a second monomer, and a third monomer, and wherein the ratio of the first monomer to the third monomer is greater than 1:1. The surface to be modified is contacted with the polymer to produce the modified surface having the polymer bound thereto. In one class of embodiments, the surface to be modified comprises a metal oxide, for example, Al₂O₃, Ta₂O₅, TiO₂, Nb₂O₅, Fe₂O₃, ZrO₂, or SnO₂.

As noted, the ratio of the first monomer to the third monomer is greater than 1:1 (e.g., greater than 2:1, greater than 5:1, greater than 50:1, greater than 100:1, or even greater than 500:1). Preferably, the ratio of the first monomer to the third monomer in the polymer is between 5:1 and 500:1. The ratio of the first monomer to the second monomer in the polymer is optionally also greater than 1:1. For example, the ratio of the first monomer to the second monomer in the polymer can be between 5:4 and 500:499. In one class of embodiments, the ratio of the first to the second to the third monomer in the polymer is between 5:4:1 and 500:499:1. The ratio of the first monomer to the sum of the second and third monomers in the polymer is optionally about 1:1.

In one example, the first monomer is a methacrylate-alkyl-phosphonate. The third monomer can comprise a polyethylene glycol or similar anti-fouling moiety; for example, the third monomer can be a polyethylene glycol methacrylate monomer or a polyethylene glycol methyl ether methacrylate monomer, e.g., one with more than four ethylene glycol repeat units. Exemplary second monomers include, but are not limited to, methacrylic acid and polyethylene glycol methacrylate monomers (e.g., a PEG-methacrylate monomer with fewer repeat units than a PEG-containing second monomer with which it is employed).

In one aspect, the copolymer includes a reactive moiety. For example, in one class of embodiments, the at least three monomers comprise four monomers, the four monomers comprising the first monomer, the second monomer, the third monomer, and a fourth monomer comprising a reactive moiety, wherein the first, second, and third monomers do not comprise the reactive moiety. The fourth monomer is optionally related to the third monomer (e.g., identical except for the presence of the reactive moiety). The fourth monomer may be present at a lower concentration than the third monomer, e.g., in embodiments in which a low density of the reactive moiety is desired on the resulting modified surface. The reactive moiety can comprise, for example, a binding moiety (e.g., nonspecific binding moiety or a specific binding moiety, e.g., one member of a specific binding pair, such as a binding moiety selected from the group of consisting of an antigen, an antibody, an binding fragment of an antibody, a polynucleotide, a binding peptide, biotin, avidin and streptavidin) or a catalytic moiety (e.g., an enzyme such as a nucleic acid polymerase, a ligase, a nuclease, a protease, a kinase and a phosphatase).

The surface can comprise an observation area or observation surface of an optical confinement.

A related general class of embodiments provides a substrate comprising a metal oxide surface and a polymer layer disposed on the surface, which layer comprises a copolymer comprising at least a first monomer comprising an alkyl phosphonate or alkyl phosphate group, a second monomer, and a third monomer, wherein the ratio of the first monomer to the third monomer is greater than 1:1. The substrate can include a zero mode waveguide array. Essentially all of the features noted for the methods above apply to these embodiments as well, as relevant, for example, with respect to types and ratios of first, second, and third monomers, inclusion of optional fourth monomers and/or reactive moieties, type of substrate, and the like.

One general class of embodiments provides methods of preparing a modified surface, in which a surface to be modified and a first surface modifying agent are provided. The first surface modifying agent comprises a polyethylene glycol moiety coupled to two or more silane groups. The surface to be modified is contacted with the first surface modifying agent, to produce the modified surface having the first surface modifying agent coupled thereto.

The first surface modifying agent optionally also comprises a reactive moiety coupled to the polyethylene glycol moiety, for example, a binding moiety (e.g., a specific or nonspecific binding moiety) or a catalytic moiety (e.g., an enzyme such as a nucleic acid polymerase, a DNA polymerase, a ligase, a nuclease, a protease, a kinase, or a phosphatase). The surface can be treated with a mixture of agents, for example, a mixture of the first surface modifying agent and a second surface modifying agent that does not comprise the reactive moiety. The density of the reactive moiety on the resulting modified surface can be controlled by controlling the ratio of the first and second (and optional third, etc.) agents. Optionally, the second surface modifying agent also comprises a polyethylene glycol moiety coupled to two or more silane groups.

In one class of embodiments, the first surface modifying agent preferentially couples to the surface rather than undergoing an intramolecular reaction. The silane groups in the surface modifying agent(s) can be essentially any of those known in the art, and in one embodiment are trimethoxysilane groups.

The surface can comprise an observation area, e.g., the observation surface of a zero mode waveguide, or an observation surface of an optical confinement. The surface optionally comprises silica, glass, quartz, fused silica, or silicon.

A related general class of embodiments provides a substrate comprising a surface to which is coupled a first surface modifying agent, which first surface modifying agent comprises a polyethylene glycol moiety coupled to two or more silane groups. Essentially all of the features noted for the methods above apply to these embodiments as well, as relevant, for example, with respect to type of first surface modifying agent, inclusion of a reactive moiety, inclusion of a second surface modifying agent, type of substrate, and the like.

Another general class of embodiments provides methods of immobilizing a desired molecule on a surface. In the methods, the surface on which the molecule is to be immobilized is provided, and a first copy of a first binding moiety is coupled to the surface. A multivalent binding intermediate which has three or more binding sites for the first binding moiety (and which is therefore capable of binding to three of more copies of the binding moiety simultaneously) is provided and bound to the first copy of the first binding moiety coupled to the surface, thereby coupling the multivalent binding intermediate to the surface. One or more of the binding sites on the multivalent binding intermediate is blocked to produce a blocked multivalent binding intermediate. A desired molecule coupled to a second copy of the first binding moiety is provided, and the second copy of the first binding moiety is bound to the blocked multivalent binding intermediate, thereby coupling the desired molecule to the multivalent binding intermediate. The various blocking and binding steps can be performed in essentially any order.

In one embodiment, the first binding moiety is biotin and the multivalent binding intermediate comprises an avidin or streptavidin. In a related class of embodiments, the multivalent binding intermediate has four binding sites for the first binding moiety, and blocking one or more of the binding sites on the multivalent binding intermediate comprises blocking two of the binding sites. In one embodiment, blocking two of the binding sites on the multivalent binding intermediate comprises providing a blocking reagent which comprises two copies of the first binding moiety (i.e., third and fourth copies) coupled by a linker, contacting the blocking reagent with the multivalent binding intermediate and permitting the two copies of the first binding moiety to occupy two of the binding sites on the multivalent binding intermediate, to produce the blocked multivalent binding intermediate, and optionally isolating the blocked multivalent binding intermediate. In another embodiment, coupling a first copy of the first binding moiety to the surface comprises coupling a first surface modifying agent to the surface, which first surface modifying agent comprises three copies of the first binding moiety, and binding the multivalent binding intermediate to the first copy of the first binding moiety and blocking two of the binding sites on the multivalent binding intermediate comprises contacting the multivalent binding intermediate and the surface-coupled first surface modifying agent and permitting the three copies of the first binding moiety to occupy three of the binding sites on the multivalent binding intermediate, to provide the blocked multivalent binding intermediate. In this embodiment, the first surface modifying agent is optionally a biotin-PEG-silane comprising three biotin moieties. Optionally, a second surface modifying agent is also coupled to the surface, which second surface modifying agent does not comprise the first binding moiety; the second surface modifying agent is optionally present in excess of the first surface modifying agent (e.g., in embodiments in which a low density of available binding sites for the first binding moiety on the resulting surface is desired).

Yet another general class of embodiments provides methods of performing a reaction involving a molecule of interest. The method includes the following steps: a) providing particles (e.g., beads) having the molecule of interest coupled to their surface; b) positioning a first subset of the particles in an observation area; c) performing the reaction; d) removing the first subset of particles from the observation area; and e) repeating steps b-d with a second subset of the particles.

The methods are optionally employed for single molecule analyses, e.g., nucleic acid sequencing by monitoring single molecule reactions in real time. Accordingly, in one class of embodiments, the molecule of interest is coupled to the surface of the particles at a density selected so that from 1 to 3 molecules of interest (preferably one) are within the observation area when the first subset of particles is positioned in the observation area. The molecule of interest can be, for example, an enzyme (e.g., a DNA polymerase), a nucleic acid (e.g., one configured to serve as a template or primer in a nucleic acid sequencing reaction), or the like. The reaction can be monitored by confocal microscopy, total-internal reflection microscopy, or essentially any other convenient technique, e.g., that permits one to optically distinguish the results of the reaction.

Accordingly, the invention also provides an optically distinguishable single molecule reaction comprising, e.g., an enzyme, nucleic acid template and/or primer bound to a particle (e.g., a bead or a nanoparticle, e.g., comprising a material of interest, e.g., a metal, a magnetic material, a quencher, a fluorescent donor, a plurality of fluorescent donors, a meta/dielectric layer, or the like). Reactions are optically distinguishable, e.g., when the reaction is present in an observation volume, zone or region that can be differentiated from surrounding regions or volumes by detecting an optical label that is found either in a reactant or in a product of the reaction. This can occur, e.g., in an optically confined observation volume such as a zero mode waveguide, or simply in close proximity to the particle. Such single molecule reactions can include, e.g., a DNA sequencing reaction. Multiple particles can be used to bring reagents or reactants into contact, e.g., a single molecule reaction can include a first reactant or reagent bound to a first particle, and a second reactant or reagent bound to a second particle, wherein the first reactant or reagent and the second reactant or reagent are different, and wherein the first and second particles are different.

Kits comprising any of the modified surfaces herein are also a feature of the invention. Such kits can additionally include packaging materials, instructions for making or using surfaces, or the like. Further, all components and methods are optionally used in operable combinations.

### BRIEF DESCRIPTION OF THE DRAWINGS

FIG. 1 is a schematic illustration of a surface having a low density of reactive moieties thereon.

FIG. 2 is a schematic illustration of a surface having two differently functionalized surface modifying agents disposed thereon (a bipodal biotin-PEG-silane and a bipodal methoxyPEG-silane), to yield a surface having a relatively low density of reactive biotin moieties.

FIG. 3 schematically illustrates an exemplary process for ensuring 1:1 stoichiometric binding of a biotinylated molecule of interest to a surface-immobilized biotin, via formation of a blocked multivalent binding intermediate.

FIG. 4 schematically illustrates an exemplary process for ensuring 1:1 stoichiometric binding of a biotinylated molecule of interest to a surface-immobilized biotinylated molecule, via formation of a blocked multivalent binding intermediate.

FIG. 5 schematically illustrates a conventional slide-based assay as compared to a bead-based assay of the invention.

Schematic figures are not necessarily to scale.

### DETAILED DESCRIPTION

The present invention is generally directed to materials and their surfaces, generally referred to hereafter as substrates, where the surfaces have been selected and/or configured to have desirable properties for a variety of applications. The invention is also directed to methods and processes for producing such surfaces, as well as methods and processes for using such surfaces in a number of different applications.

Of particular interest with respect to the present invention are substrates and surfaces that possess selective molecular binding or coupling characteristics, e.g., through the selective inclusion of molecular binding moieties thereon, and the use of such surfaces to selectively bind desired molecules to the surfaces in a selective fashion. Of still greater interest is the use of such surfaces when they are selectively coupled to chemically and/or biologically active molecules for use in chemical and/or biochemical processes, such as in preparative operations and/or analytical operations.

Although the invention has broad applicability, as will be apparent from the ensuing disclosure, in one aspect, the surfaces have a low density of reactive groups. Optionally, the surfaces include a single reactive group, in preferred cases, an enzyme such as a nucleic acid polymerase, within an area that is being observed and/or monitored, giving the observer a real-time understanding of the reactions catalyzed by that single enzyme, e.g., DNA synthesis. Such systems are particularly useful in template dependent analysis, or sequencing, of nucleic acids.

Also of interest with respect to the present invention are substrates and surfaces that have been passivated to render them non-reactive, reducing non-specific binding to the surfaces.

### Substrates and Surfaces

Generally

As alluded to above, the ability and/or propensity of surfaces to interact on a molecular level with their surroundings is of particular interest in the chemical and biological sciences and industries exploiting those sciences. For example, past efforts at manipulation of the reactive groups present on surfaces have focused primarily on one extreme or another. In particular, a number of applications benefit from maximizing the density of molecules bound to a particular surface by maximizing the number of reactive groups on that surface, e.g., high density binding. In other applications, the desired goal has been to exclude virtually all binding or other coupling interactions, including adsorption, between a surface and materials exposed to those surfaces, to create an inert surface for the given application, by capping or otherwise masking reactive groups on the surface.

For example, in the case of biologically reactive surfaces, DNA array technology has focused upon binding as many active polynucleotide probes within a given area as possible, so as to maximize the signal generated from hybridization reactions with such probes. Likewise, affinity surfaces employing, e.g., antibodies, have similarly focused upon increasing the density of binding groups on a surface to improve sensitivity. Alternatively, in a number of other applications, past efforts have been directed at effectively neutralizing the binding effects of surfaces to minimize or eliminate the surface's interaction with the chemical or biochemical environment. For example, the field of microfluidics, and particularly the capillary electrophoresis art, is replete with examples of researchers identifying coating materials or other surface treatments that are intended to mask any functional groups of fused silica capillaries to avoid any molecular associations with those surfaces. In one aspect, the present invention provides methods of preparing modified surfaces that can be used to passivate surfaces, minimizing the surfaces' interactions with the environment (e.g., minimizing or eliminating nonspecific binding to the surfaces).

For certain applications, however, surfaces that are neither intended to maximize nor completely eliminate reactive chemical groups on a given surface, but that instead have a selected relatively low density of reactive groups on the surface, are desirable. See U.S. patent application Ser. No. 11/240,662, entitled “REACTIVE SURFACES, SUBSTRATES AND METHODS OF PRODUCING AND USING SAME” by Roitman et al., filed Sep. 30, 2005, and international patent application PCT/US2006/38243 Sep. 29, 2006, which describe surfaces having low densities of reactive groups and methods of producing such surfaces. In addition, one aspect of the present invention is directed at providing a surface with reactive groups, e.g., at a selected relatively low density, and the use of such surfaces in a number of valuable applications. As will be appreciated, the nature of reactive groups (also called reactive moieties herein) does not imply or require a group capable of covalent linkage with another group, but includes groups that give rise to other forms of interaction, including hydrophobic/hydrophilic interactions, Van der Waals interactions, and the like. As such, surface reactivity, as generally described herein, includes, inter alia, association by covalent attachment and non-covalent attachment, e.g., adsorption.

Although, for ease of discussion, the substrates and surfaces are generally described herein in terms of planar solid substrates, it will be appreciated that the methods, processes, surfaces, etc. of the invention are applicable to a variety of different substrate types where the properties of reactive surfaces of the invention may be useful. In particular, such surfaces may comprise planar solid surfaces, including inorganic materials such as silica based substrates (i.e., glass, quartz, fused silica, silicon, or the like), other semiconductor materials (i.e., Group III-V Group II-VI or Group IV semiconductors), metals or metal oxides (e.g., aluminum or aluminum oxide), as well as organic materials such as polymer materials (i.e., polymethylmethacrylate, polyethylene, polypropylene, polystyrene, cellulose, agarose, or any of a variety of organic substrate materials conventionally used as supports for reactive media). In addition to the variety of materials useful as substrates, it will be appreciated that such materials may be provided in a variety of physical configurations, such as microparticles, e.g., beads, nanoparticles, e.g., nanocrystals, fibers, microfibers, nanofibers, nanowires, nanotubes, mats, planar sheets, planar wafers or slides, multiwell plates, optical slides including additional structures, capillaries, microfluidic channels, and the like.

In operation, selective and limited reactivity of the surfaces of the invention is aimed at providing, in a limited fashion, a particular desired molecule or type of molecule of interest, typically a selected reactive molecule of interest, on a surface, e.g., a particular enzyme, nucleic acid, or the like, while preventing binding of the molecule of interest and/or other potentially interfering molecules elsewhere on the surface. For preferred applications, the desired result is a surface that includes a relatively low density of the selected reactive molecule surrounded by an otherwise non-reactive surface. Although discussed in terms of a molecule or type of molecule of interest, it will be appreciated that mixed functionality surfaces are also encompassed within the scope of the invention, including, e.g., two, three, four, or more different molecules or types of molecules of interest.

Thus, as used herein, the terms “reactive” and “non-reactive” when referring to different groups on the substrate surfaces of the invention refers to (1) the relative reactivity or association of such surface components with a given molecule of interest, and preferably also refers to (2) the relative reactivity or association of such surface components with other reagents in a given application of such surfaces, where such reagents may interfere with such applications, such as labeled reactants and or products that might interfere with detection, as well as inhibitors or other agents that would interfere with the progress of a reaction of interest at the reactive portion of the surface or elsewhere.

In terms of the first aspect of such reactivity, the reactive portions or groups on the surfaces will typically have 10 times greater affinity for the molecule of interest, preferably more than 100 times greater affinity and more preferably at least 1000 times greater affinity for the molecule of interest than the non-reactive surface. As such, it will be appreciated that the level of association between the molecule of interest and the reactive surface will be substantially greater than with the non-reactive surface under uniform conditions, e.g., more than 10 times greater, more than 100 times greater and preferably more than 1000 times greater. Such greater association includes greater frequency and/or greater duration of individual associations.

In terms of the second aspect of surface reactivity or non-reactivity, in many cases, such reactivity is coincident with the first aspect. In particular, where an enzyme constitutes the reactive portion of the surface, it will generally have a high affinity for its substrate, and thus associate with such substrate at a much greater level than the non-reactive portion, e.g., as described above. However, in some cases, the “reactive” portion of the surface may not include an ability to associate with certain potential interfering molecules. In such cases, the terms adsorptive and non-adsorptive also may be used. Nonetheless, it is desirable to prevent such interfering molecules from associating with the remainder of the surface. As such, the non-reactive surface may be defined in terms of its reactivity with such interfering components.

Because the primary source of undesirable interference for many applications lies in the non-specific interaction of reagents with the non-reactive portions of the surface, rather than at the desired reactive portion, the non-reactive surface in such cases may generally be characterized by an association equilibrium constant between the non-reactive group and a particular interfering molecule that is preferably 10 fold lower than the association equilibrium constant of the reactive surface(s) with the reactive molecule(s), and preferably 100 fold (or more) lower. The association reaction for the non-reactive surface is also characterized by a low activation barrier, such that the kinetics of the corresponding dissociation reaction are expected to be fast, with average binding time preferably at least 10 fold lower than the significant timescales of the measurement process of the application, and preferably 100 fold lower or more.

As will be appreciated, the characteristics of such non-reactive and reactive surfaces will typically depend upon the specific application to which the surface is to be put, including environmental characteristics, e.g., pH, salt concentration, and the like. In particularly preferred aspects, environmental conditions will typically include those of biochemical systems, e.g., pH between about 2 and about 9, and salt levels at biochemically relevant ionic strength, e.g., between about 0 mM and 100 mM.

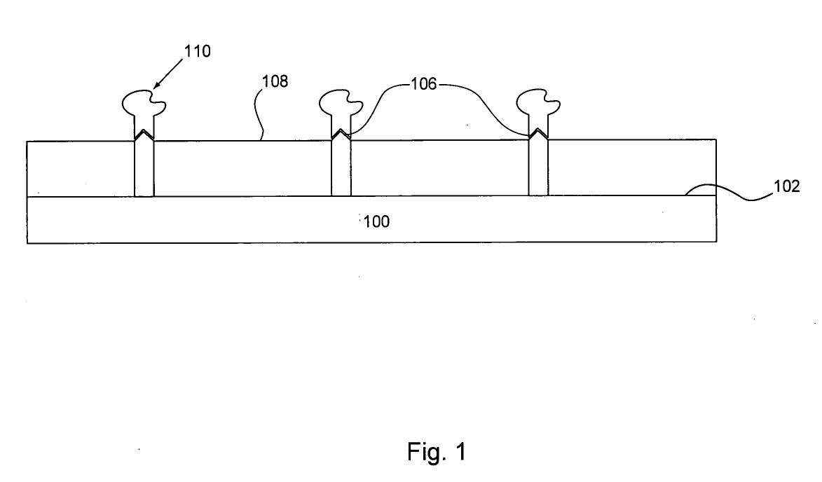

FIG. 1 provides a simplified schematic illustration of the low density reactive group surfaces of the invention, in block diagram form. As shown, a substrate 100 includes a surface 102. (As described in U.S. patent application Ser. No. 11/240,662, the surface 102 is optionally derivatized to provide an overall active surface, or the substrate may inherently possess an overall reactive surface.) Surface 102 is treated to provide a surface that includes reactive groups 106 coupled to the reactive surface 102 at relatively low densities. As noted, these reactive moieties are preferably disposed upon or among an otherwise neutral or non-reactive surface 108. In particularly preferred aspects, the reactive groups 106 may include, or be further treated to include additional reactive groups, e.g., catalytic components, such as enzymes 110, or the like, as also shown in FIG. 1.

One important advantage of the surfaces of the invention is the optional provision of relatively isolated reactive groups. Isolation of reactive groups provides the ability to perform and/or monitor a particular reactivity without interference from adjacent reactive groups. This is of particular value in performing single molecule reaction based analyses, where detection resolution necessitates the isolation, e.g., to be able to optically distinguish between reactive molecules (optical isolation), electrochemically distinguish between reactions at different reactive molecules (electrochemical isolation) or where chemical contamination from one reaction at one location may impact reaction at an adjacent location (chemical isolation).

An additional advantage of the surfaces of the invention is the ability of the surface, or, for embodiments in which the surface includes reactive moieties, the remainder of the surface, to be inert to coupling with potentially interfering molecules, e.g., fluorescent analytes or products. In particular, while binding of a few selected molecules is desirable for a set of applications, uncontrolled or nonspecific binding the remainder of the surface is often highly undesirable. By providing the desired reactive groups only at a selected, relatively low density, which themselves comprise a moiety having a desired reactivity, or which in some cases are reacted with another molecule having the desired reactivity, one can selectively treat the remainder of the surface as necessary to render it effectively neutral to unwanted binding, thus substantially reducing or eliminating such unwanted binding elsewhere on the surface. In accordance with preferred aspects of the invention, both the provision of selected reactive groups and the provision of non-reactive groups over the remainder of the surface to reduce such unwanted surface interactions are accomplished in the same process step or steps.

Density

In accordance with certain embodiments of the invention, the low density of the selected desired reactive moieties or chemical groups on a surface is designed to provide a single reactive moiety within a relatively large area for use in certain applications, e.g., single molecule analyses, while the remainder of the area is substantially non-reactive. Typically, this means that any reactive groups otherwise present upon the remainder of the surface area in question are capped, masked, or otherwise rendered non-reactive. As such, low density reactive groups are typically present on a substrate surface at a density of reactive groups of greater than 1/1×10⁶ nm² of surface area, but less than about 1/100 nm². In more preferred aspects, the density of reactive groups on the surface will be greater than 1/100,000 nm², 1/50,000 nm², 1/20,000 nm² and 1/10,000 nm², and will be less than about 1/100 nm², 1/1000 nm², and 1/10,000 nm². For certain preferred applications, the density will often fall between about 1/2500 nm and about 1/300 nm², and in some cases up to about 1/150 nm².

Observation Areas

In certain particularly preferred aspects, the methods and surfaces of the invention provide reactive groups on a surface at a density such that one, two, three or a few reactive groups are present within an area that is subject to monitoring or observation (an “observation area”). By providing individual or few reactive groups within an observation area, one can specifically monitor reactions with or catalyzed by the specific individual reactive group. Such observation areas may be determined by the detection system that is doing the monitoring, e.g., a laser spot size directed upon a substrate surface to interrogate reactions, e.g., that produce, consume or bind to fluorescent, fluorogenic, luminescent, chromogenic or chromophoric reactants, or fiber tip area of an optical fiber for optical monitoring systems, a gate region of a chemical field effect transistor (ChemFET) sensor, or the like, or they may be separately defined, e.g., through the use of structural or optical confinements that further define and delineate an observation area.

One example of a particularly preferred observation area includes an optical confinement, such as a zero mode waveguide (ZMW). Zero mode waveguides, as well as their use in single molecule analyses, are described in substantial detail in U.S. Pat. No. 6,917,726, which is incorporated herein by reference in its entirety for all purposes. Such ZMWs have been exploited for use in single molecule analyses, because they can provide observation volumes that are extremely small, e.g., on the order of zeptoliters. In such cases, the observation area will generally include the cross sectional area of the observation volume, and particularly that portion of the observation volume that intersects the surface in question (i.e., the observation surface).

In certain preferred aspects, the invention provides one or only a few reactive groups on the bottom surface of the waveguide. In such cases, the density is measured by the number of reactive groups divided by the surface area of the bottom surface of the waveguide. Thus, purely for purposes of exemplification, where a circular waveguide has a radius of 10 nm, and includes a single reactive molecule immobilized on its bottom surface, the density of reactive groups would be approximately 1/314 nm². Thus, in terms of zero mode waveguides or other observation areas, and for purposes of example, it will be appreciated that reactive molecules present at a density of one, two, three or up to 10 reactive molecules in an area having a radius of between about 10 and about 100 nm, or areas from 314 nm to about 31,416 nm², respectively (i.e., larger numbers of molecules in larger areas), are encompassed by the densities herein described. In preferred aspects, one, two or three molecules per observation area is generally preferred.

In many cases, ZMWs are provided in arrays of 10, 100, 1000, 10,000 or more waveguides. As such, immobilization of a single reactive group, e.g., an enzyme, within each and every ZMW would be difficult. However, dilution based protocols, when combined with the surfaces of the invention. while producing some ZMWs that are not occupied by an enzyme, will generally result in the majority of occupied ZMWs (those having at least one enzyme molecule immobilized therein) having only one or the otherwise desired number of enzymes located therein. In particular, in the case of ZMWs having reactive molecules like enzymes located therein, typically, more than 50% of the occupied ZMWs will have a single or the desired number of reactive molecules located therein, e.g., a particular type of enzyme molecule, preferably, greater than 75%, and more preferably greater than about 90% and even greater than 95% of the occupied ZMWs will have the desired number of reactive molecules located therein, which in particularly preferred aspects may be one, two, three or up to ten reactive molecules of a given type. As noted elsewhere, in some circumstances different reactive molecules may also be provided at a desired density to provide a mixed functionality surface. In accordance with the present invention, depending upon the types of reactive groups being referenced, e.g., catalytic or binding, it will be appreciated that the determination of density may be applied on a single occupied ZMW, or upon multiple ZMWs in an array.

Specific Reactive Groups

The reactive groups or moieties present on the surfaces of the invention include a wide range of different types of reactive groups having chemical and/or biological activity, which are coupled (covalently or non-covalently) to a surface of a material or substrate, either by exogenous addition or which inherently are present on such surface. These reactive groups include groups on a surface that possess binding activity for other chemical groups, e.g., the ability to bind another chemical moiety through specific or non-specific interactions, through covalent attachment, Van der Waals forces, hydrophobic interaction, or the like. Provision of a wide range of reactive groups on surfaces is readily understood in the art, and includes, for example, ionic functional groups, polyionic groups, epoxides, amides, thiols, hydrophobic groups, e.g., aliphatic groups, mono or polycyclic groups, and the like, e.g., as generally used in reverse phase and/or hydrophobic interaction chromatography (HIC), staudinger ligation groups (see, e.g., Lin et al., J. Am. Chem. Soc. (2005), 127:2686-95), Click chemistry coupling using chemoselective azide-acetylene linkages (See, Deveraj et al., JACS 2005, 127:8600-8601; Lummerstorfer et al., J. Phys. Chem. B (2004) 108:3963-3966, and Collman et al., Langmuir (2004) 20:1051-1053, each of which is incorporated herein by reference in its entirety for all purposes) and other groups that associate or are capable of being coupled with other groups in a non-specific fashion. Additionally, use of specific binding groups on surfaces, e.g., groups that specifically recognize a complementary binding partner has been described, including, e.g., complementary nucleic acid pairs, antibody-epitope pairs, binding peptides that recognize specific macromolecular structures, e.g., recognition sequences in proteins, peptides or nucleic acids, lectins, chelators, biotin-avidin (or biotin-streptavidin) linkages, and the like.

Identification of the number and/or density of reactive groups may generally be ascertained through the use of a reporter molecule, which in many cases may be the reactive group itself. In particular, and by way of example, one can ascertain the number of enzyme molecules coupled to a surface area by assaying for the activity of that enzyme. Likewise, other reactive groups may be quantified through other methods, e.g., titration, coupling of labeling groups, or the like.

As used herein, both reactive groups and non-reactive groups envision an environment in which the surfaces are to be applied, and in which the reactivity, or non-reactivity, is evident. As will be appreciated, different groups may be reactive in certain environments and non-reactive in others, and the invention, as broadly practiced, envisions applicability in a wide range of different environments. For ease of discussion, and in preferred aspects, the surfaces of the invention are most often to be applied in biological or biochemical reactions, and as such are subjected to appropriate environments. Such environments typically include aqueous systems having biochemically relevant ionic strength, that range in pH between about 2 and about 9, and preferably between about 5 and about 8, but may vary depending upon the reactions being carried out.

In certain preferred aspects, the reactive chemical groups also include groups having catalytic activity, e.g., the ability to interact with another moiety to alter that moiety other than through binding, i.e., enzymatic activity, catalytic charge transfer activity, or the like. In particularly preferred aspects, the active chemical groups of the invention include chemical binding groups, and optionally and additionally, catalytic groups, where the binding group is used to couple the catalytic group to a given surface in accordance with the invention. For example, an enzyme or other catalytic group may be coupled to a surface via an intermediate binding or linker group that is, in turn, coupled directly to a reactive group that is disposed upon the surface material at a desired density.

A number of different reactive groups may be employed in accordance with the invention, and may to some extent depend upon the surface being used, and whether the reactive group is intended to provide a low-density general or non-specific binding or associative function, a low-density specific binding function, or a low density catalytic function.

For example, for silica based surfaces, e.g., glass, quartz, fused silica, silicon or the like, reactive groups may be provided by silane treatment of the surface, e.g., using epoxysilane, aminosilane, activated carboxylic acid silane, isocyanatosilane, aldehyde silane, mercaptosilane, vinyl silane, hydroxyterminated silanes, acrylate silane, trimethoxysilane, and the like. Such treatments may yield the reactive groups, e.g., in terms of low density, non-specific associative groups, or they may result in or be further treated, to provide a specific binding group or catalytic group, as the ultimate reactive group. Alternatively or additionally, other inorganic or organic reactive groups may be provided upon a surface. In the case of inorganic surfaces like silica based substrates, such additional materials may be coupled to the surface via an intermediate chemical coupling, e.g., using silane chemistry, i.e., as described above. These additional materials may include small molecules, e.g., ionic groups, metal ions, small organic groups, as well as larger or polymeric/oligomeric molecules, e.g., organic polymers. For ease of discussion, polymer and oligomer are used interchangeably herein to refer to molecules that include multiple subunits of similar chemical structure.

In particularly preferred aspects, a longer linker molecule, and preferably an organic linker molecule, may be used to link the reactive group to the surface to provide further flexibility to the overall linkage, e.g., by providing greater spacing between the surface and reactive group. In particular, polymeric or oligomeric chains that bear the desired reactive group at one end may be linked at the other end to the surface, e.g., via silane linkage in the case of a glass surface. By selecting different types and lengths of polymer linkers, one can further adjust the properties of the surface, e.g., relative hydrophobicity of different groups/areas, relative distance to the surface, overall or local surface charge, and the like. Examples of useful polymer linkers include, e.g., cellulosic polymers (such as hydroxyethyl-cellulose, hydroxypropyl-cellulose, etc.), alkane or akenyl linkers, polyalcohols (such as polyethyleneglycols (PEGs), polyvinylalcohols (PVA)), acrylic polymers (such as polyacrylamides, polyacrylates, and the like), polyethylene polymers (such as polyethyleneoxides), biopolymers (such as polyamino acids like polylysine, polyarginine, polyhistidine, etc.), other carbohydrate polymers (such as xanthan, alginate, dextrans), synthetic polyanions or polycations (such as polyacrylic acid, carboxyl terminated dendrimers, polyethyleneimine, etc.) and the like. Again, depending upon the type of linker used, the linker may further include a desired reactive group coupled to it.

While described generally in terms of application of a reactive group to the surface, it will be appreciated that the active group may be applied to the surface as an inactive or less reactive precursor to the desired reactive group, and subsequently activated to yield the desired reactive group. In particular, the reactive groups may be provided as photo, thermally or chemically activatable precursor groups, e.g., bearing a photolytic capping group, a temperature sensitive capping group or an acid or base labile capping group, blocking the reactive moiety of interest. The group may then be selectively activated, e.g., through the use of photo, thermal or chemical treatment to yield the desired surface. A variety of such groups are known in the art and are described in, e.g., Guillier, et al., Linkers and Cleavage Strategies in Solid Phase Organic Synthesis and Combinatorial Chemistry, Chem. Rev. 100:2091-2157 (2000).

As noted above, the reactive groups on a surface may be comprised of the aforementioned specific or non-specific binding moieties, or may include catalytic groups that are coupled to the surface, either directly to the surface, through the above mentioned specific or non-specific binding or associative groups, that are, in turn, coupled directly or indirectly to the surface, or through additional specific or non-specific binding groups coupled to the surface. Catalytic groups may include catalytic chemicals, e.g., catalytic metals or metal containing compounds, such as nickel, zinc, titanium, titanium dioxide, platinum, gold, or the like. In preferred aspects, however, the catalytic moieties present at a desired (e.g., low) density on the surfaces of the invention comprise bioactive molecules including, e.g., nucleic acids, nucleic acid analogs, biological binding compounds, e.g., peptides or proteins, biotin, avidin, streptavidin, etc., and enzymes. In the case of nucleic acids or nucleic acid analogs, such surfaces find use in a variety of specific binding assays, e.g., to interrogate mixtures of nucleic acids for a nucleic acid segment of interest (See, e.g., U.S. Pat. Nos. 5,153,854, 5,405,783, and 6,261,776). Likewise, binding proteins and peptides are often useful in interrogating biological samples for the presence or absence of a given molecule of interest. Typically such proteins or peptides are embodied in antibodies or their binding fragments or binding epitopes of such antibodies. In particularly preferred aspects, the surfaces of the invention bearing the catalytic groups comprise an enzyme of interest and are used to monitor the activity of that enzyme. A wide variety of enzymes are regularly monitored and detected in biological, biochemical and pharmaceutical research and diagnostics. Examples of preferred enzymes include those monitored in genetic analyses like DNA sequencing applications, such as polymerases, e.g., DNA and RNA polymerases, nucleases (endo and exonucleases), ligases, and those involved in a variety of other pharmaceutically and diagnostically relevant reactions, such as kinases, phosphatases, proteases, lipases, and the like.

With respect to immobilization of enzymes on surfaces in accordance with the invention, yet a further advantage of the surfaces of the invention stems from the combined advantages set forth elsewhere herein. In particular, in selectively immobilizing biomolecules, like enzymes, through specific linkages, and rejecting their adsorption elsewhere on the surface, the activity of the biomolecules present on the surface can be more selectively preserved, where mere adsorption may have yielded a significant population of inactive or less active molecules. Thus, on the resulting surface, the biomolecules present, while optionally present at low density, will nonetheless be present at a relatively high specific activity (e.g., number of active biomolecules of interest vs. total number of biomolecules present).

In the case of certain catalytic reactive groups, e.g., enzymes, the density of such reactive groups further envisions the density of active molecules, as opposed to immobilized inactive molecules. For example, in the case of enzymes immobilized on a surface at a relatively low density, such density will typically include an allocation for the specific activity of the immobilized enzyme, e.g., the efficacy of the immobilization process. Thus, where the immobilization process yields only 50% viable or active enzymes, the overall density of enzyme molecules, active and otherwise, will generally be 2× the density of active molecules. Accordingly, in ascertaining the desired density of such reactive groups, it is often desirable to assess the relative efficacy of the immobilization process in depositing active molecules. Optionally, the methods provide specific activities (fraction of immobilized enzyme having activity) of greater than 20%, greater than 30%, more preferably, greater than 50% and in still more preferred aspects, greater than 75%, and in some cases greater than 90%.

In contrast to the low density of desired reactive groups on the substrates of the invention, it is also typically preferred that the remainder of the surfaces in question be non-reactive. As noted previously, such non-reactivity includes a substantially lower affinity for a molecule of interest as compared to the reactive groups, but additionally, preferably includes a lack of excessive binding or association with molecules that would potentially interfere with the end-application of the surface. For example, where additional catalytic groups are to be coupled to a desired low density population of desired reactive groups on a surface, it is generally desired that such catalytic groups not associate substantially with the remainder of the surface, either specifically or non-specifically. Likewise, in applications where additional chemical groups will be exposed to the surfaces of the invention, it will generally be desired that the remainder or non-reactive surface not catalyze reactions with such materials or bind or otherwise associate with the materials that might provide adverse or noisy signals that do not correspond to the reactions of the reactive groups of interest. Non-reactivity is similarly defined for passivated surfaces that do not include reactive groups; i.e., such surfaces do not catalyze reactions or bind or otherwise associate with such materials.

In the case of fluorescent single molecule assays, one particular desire is to avoid excessive (e.g., in duration and/or frequency), nonspecific binding or association or “sticking” of unreacted fluorescent reagents or fluorescent products with the surface other than with the reactive groups of interest, e.g., an enzyme, as such associations can lead to erroneous signal production, background signal noise, and signal noise build-up over time. In general, it will be desired that non-specific association of compounds with the non-reactive portion of the surface (or with a non-reactive surface) will be comparable to the rate of diffusion of such compounds in solution. Rephrased in terms of labeled compounds being observed in observation areas or optical confinements, signal resulting from the non-specific association of compounds with the non-reactive surface will typically be on the same or similar order, e.g., less than 100 times such diffusion based signals and preferably less than 10 times such diffusion based signals (in either or both of duration and frequency), as signal resulting from random diffusion of such compounds into and out of the observation area or volume of fluid for a given analysis. In terms of fluorescent compounds or other signal generating compounds that might potentially interfere with the desired application, it will generally be desirable that any signal resulting from association of such compounds with the non-reactive surface (referred to herein as “non-specific signal generation”), will be at least 10 fold lower than signal generated by the reactive groups, preferably more than 100 fold less, and still more preferably, more than 1000 fold less than signal resulting from action of the reactive molecules (“specific signal generation”), e.g., desired enzyme activity. Such reductions in non-specific signal generation includes reductions in either or both of frequency or duration, e.g., reductions in the number of signal events or a reduction in the aggregate amount of signal emanating from such non-specific signal generation.

A variety of non-reactive groups may be employed upon the remainder of the surface that will, again, depend upon the environment to which the surface will be subjected. In general, however, terminal hydroxyl groups, methyl groups, ethyl groups, cyclic alkyl groups, methoxy groups, hydroxyl groups, e.g., in non-reactive alcohols and polyols, inactivated carboxylate groups, ethylene oxides, sulfolene groups, hydrophilic acrylamides, and the like are optionally employed as non-reactive groups.

Layered Surfaces/Thickness

As repeatedly described above, the reactive groups, as set forth above, may be coupled directly to the surfaces of the substrates or coupled through one or more intermediate linking groups that provide one or more intermediate molecular layers between the desired reactive group and the inherent or native surface of the substrate material. Restated, each component of the surface, reactive or non-reactive, may result from one or more layers of components to provide the desired resulting surface component.

For example, in its simplest form, both reactive and non-reactive groups may be coupled directly to a substrate's native surface to yield the low-density reactive surfaces of the invention. Alternatively, one or more layers of linking groups may be added to the surface to yield a layered surface, to which the reactive and non-reactive groups are then coupled to yield the desired surface. In either of these cases, the process for apportioning reactive and non-reactive groups on the surface occurs in the deposition of the final layer.

In still more complex configurations, apportionment of the reactive and non-reactive groups on the final surface layer may occur in the selection and deposition of earlier layers on the surface. In other words, a first low-density reactive layer may be used to dictate the deposition of a subsequent or desired low-density reactive layer. By way of example, a first layer that includes a low density of non-specific binding groups may be used as a template for the deposition of a subsequent layer with a low density of catalytic groups, e.g., where the catalytic groups couple to the binding groups. In still further aspects, such apportionment may take place over multiple layers, to more finely tune the deposition process. For example, a first apportioned layer, e.g., including a mixture of binding groups and nonbinding groups, may underlie an additional layer that includes a further apportionment. Such complex layers are also particularly useful in depositing surfaces according to the invention that include a number of different types of reactive groups on an otherwise non-reactive surface, e.g., different enzymes, different nucleic acids, different antibodies, and the like.

In accordance with the foregoing, in some cases, a surface's inherent properties may permit coupling of reactive or intermediate groups thereto, while in many cases, the surfaces must first be derivatized to provide reactive groups, either for use as such, or for further coupling to intermediate linking groups. In many cases, the derivatization process may be concurrent with the coupling of reactive groups by providing the desired reactive group as a constituent of the derivatizing chemical. In such cases, the derivatizing agent bearing the reactive group of interest is coupled to the surface at a relatively low density. Typically, and as set forth in greater detail in U.S. patent application Ser. No. 11/240,662, this is accomplished by providing the derivatizing agent bearing the reactive group of interest in an appropriate ratio with derivatizing agent that, other than its ability to modify the surface, is substantially non-reactive.

In other configurations, the entire surface may be derivatized using any of the aforementioned reactive groups to provide a reactive surface to which an intermediate linking group may be coupled. In such cases, the intermediate linking group, which is provided in a ratio of linking group bearing a reactive group of interest and a non-reactive linking group is then contacted with the reactive surface to provide the desired density of reactive groups of interest on the ultimate surface. As will be appreciated, an intermediate reactive or coupling group may be provided at a higher density than the density at which the desired, final reactive group is provided, depending upon the level of coupling of that final group to the intermediate group. For example, if it is anticipated (or even planned) that the final reactive group will couple to the intermediate coupling group at a rate of 1 linkage for every ten intermediate groups, then such intermediate reactive groups may be present at a level 10 times higher. Typically, when employing such intermediate reactive groups, their density will be between about 1 and about 1000 times greater than the final reactive group, often between about 1 and about 100 times, and in some cases from 1 to about 10 times greater than the density of the final reactive group, e.g., an enzyme. Additional details can be found in U.S. patent application Ser. No. 11/240,662.

### Methods of Preparing Substrates and Surfaces

A number of methods can be used to prepare surfaces of the invention. In one aspect, robust reactive or non-reactive PEG-dense surfaces are prepared using branched PEG-silanes. In another aspect, surfaces are modified with copolymers including alkyl phosphonates or alkyl phosphates to produce reactive or non-reactive surfaces. In one aspect, the number of binding sites on a multivalent linker molecule such as streptavidin is reduced, to facilitate formation of surfaces with a low density of reactive groups. Methods of preparing modified surfaces are a feature of the invention, as are substrates comprising surfaces prepared or produced by any of the methods.

Modification with Multipodal PEG Silanes

Modification of surfaces with PEG-silanes is described in U.S. patent application Ser. No. 11/240,662 and 11/731,748 “ARTICLES HAVING LOCALIZED MOLECULES DISPOSED THEREON AND METHODS OF PRODUCING SAME” by David R. Rank et al.; see also WO2007/123763 (having the same title and inventors). Modification with PEG-silanes provides a convenient way to control the properties of surfaces, particularly silicon oxide and other oxide surfaces. In addition, it provides a convenient technique for selectively modifying one material in a hybrid substrate while leaving another material unchanged (e.g., modifying silicate surfaces and not metal surfaces in ZMWs); see 11/731,748 and WO2007/123763.

The hydrolytic stability of surface modification with PEG reagents using silanes as attachment points can, however, be improved. The Si—O—Si bond is susceptible to hydrolysis, and moreso in the case of PEG-silanes as compared to lower molecular weight silanes. Without limitation to any particular mechanism, the bulkiness of the PEG chain (sometimes called mushroom conformation) may create a region of exclusion around each grafted chain, such that the silane end group does not have the opportunity to form extended cross-linked three-dimensional networks, in contrast to surface-bound lower silanes (e.g., aminopropyl silane) which do form such networks. That is, in a molecule such as silane-PEG24-biotin or silane-PEG24-methoxy, the PEG chain may hinder the ability of the silane reactive group to reach the surface and the ability of one silane group to react with another silane in solution; the PEG chain may especially inhibit the ability of silane groups from different molecules to both cross-link with each other and attach to the surface. (Moreover, once a single Si—O—Si bond has formed between two PEG-monosilane molecules, not only are the silane groups buried in the middle of a molecule twice as long as the original PEG chain, but also close proximity between the coupled silane groups may result in capping by formation of two additional Si—O—Si bonds between the molecules, rather than bonds with a third molecule or with the surface.) Low molecular weight silanes do not experience such hindrance.

The relatively low hydrolytic stability of silanes is particularly detrimental in chromatography separation columns at high temperatures or under strongly basic conditions. In order to improve column stability, polydentate coatings have been proposed (e.g., Blaze™ multiple point bonding columns by Selerity Technologies Inc. (Salt Lake City, Utah) and Restek (State College, Pa.). In general, the strategy pursued by manufacturers of such columns has been to create multiple attachment points to the substrate as a way of stabilizing the coatings. Similarly, MicroSurfaces, Inc. (Minneapolis, Minn.) uses a multi-arm PEG to graft multiple points to a chlorinated silicon surface, resulting in greater stability than for single PEG chains. See also Antonucci et al. (2005) “Chemistry of silanes: Interfaces in dental polymers and composites” J. Res. Natl. Inst. Stand. Technol. 110:541-558 for a description of multipodal silanes.

In single molecule detection such as in ZMWs, the issue of surface robustness is of critical importance. Since only one molecule is under observation, the ability to conduct an experiment over any length of time hinges on the ability of the linker molecule to provide a stable platform to the sensing molecular complexes over an extended time frame.

According to one aspect of the invention, robust dense PEG surfaces on silica and other oxide substrates, e.g., for single molecule fluorescence detection, can be prepared using branched or other bi- or multipodal PEG silanes. Compounds having two or more silane groups coupled (typically covalently, either directly or indirectly via other PEG moieties or other chemical moieties) to at least one PEG moiety are employed to modify surfaces, such that a resulting modified surface has more than one silane group anchoring the PEG on the surface. As used herein, a “polyethylene glycol” (PEG) or a “PEG moiety” is or comprises an oligomer or polymer of ethylene oxide that includes two or more subunits (e.g., 10 or more, 20 or more, 30 or more, 40 or more, 50 or more, 60 or more, even up to 100 or more monomers). PEGs include, e.g., linear, branched, and dendritic PEGs. A “silane group” comprises a tetrahedral Si atom. Silane groups of particular interest in the context of the present invention include groups of the form —SiX₃ where X is Cl, OH, or OR (where R is an alkyl group or hydrocarbon group).

It will be evident that there are a number of approaches to making such robust, dense PEG surfaces. In one exemplary class of embodiments, to produce a biocompatible surface, an end-capped silane-PEG-silane or a branched (more than two arms) all silane-terminated PEG is synthesized and employed. An exemplary branched all silane terminated PEG is:

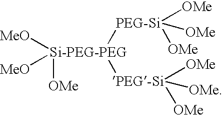

Other branched or dendritic structures with the silane groups at or toward the ends of the arms are also contemplated (e.g., 4, 6, or 8 arm PEGs, e.g., with trimethoxysilane groups on the ends of the arms).

In another exemplary class of embodiments, the surface is modified with a compound (surface modifying agent) that has more than one silane linker on one end of the PEG chain and a single or multiple non-reactive or reactive functional group(s) on the other end(s). For instance, using a diene, mono-bromoalkyl molecule as shown below, it is possible to couple two molecules of trimethoxysilane via hydrosilylation to a single PEG molecule.

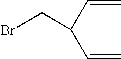

The Br group can be made to react to a PEG and the bifunctional vinyl groups can be hydrosilylated to yield a molecule with the structure:

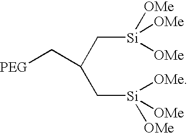

The PEG can be capped with a non-reactive moiety (e.g., methoxyPEG), or it can be coupled to a reactive moiety such as those described herein. For example, the PEG can be PEG-X, where X is a carboxyl, epoxy, amine, biotin, isocyanato, alcohol, aldehyde, photocrosslinkable, N3, or redox group.

Another exemplary branched PEG silane is:

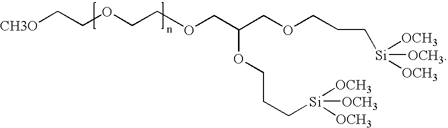

In this compound, n is optionally 20-25. (It will be evident that similar compounds can be employed that include a reactive group instead of the methoxy group.) However, it is possible that the above compound can undergo an intramolecular reaction in which the methoxysilane groups react with each other, deactivating the molecule, as shown in the following structure:

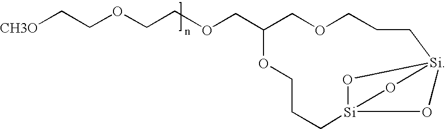

In one aspect, a moiety that sterically hinders intramolecular reaction between silane groups (e.g., by preventing the molecule from assuming a conformation in which the silane groups are in close proximity) is employed to couple the silane groups to the PEG moiety. Exemplary silanes that have more than one silane moiety at one end of the PEG chain, coupled via a sterically hindered moiety to help maximize attachment to the surface and minimize self-condensation, include, but are not limited to, the following compounds:

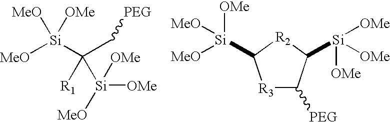

where

### R₁ is CH₃, CH(CH₃)₂, OH, NH₂, O—CH₃, or PEG

### R₂ is CH₂, S, NH, O, N—R₄, or PEG

### R₃ is CH₂, S, NH, N—R₄, or PEG

R₄ is H, CH₃, CH₂CH₃ . . . etc. (an alkyl group).

In general, a PEG including two or more silane groups for use in the methods preferably preferentially couples to the surface rather than undergoing an intramolecular reaction. For example, in some embodiments, when the bipodal or multipodal PEG is used to modify a surface, less than 25% of molecules undergo an intramolecular reaction instead of coupling to the surface (or remaining available to couple to the surface) under standard reaction conditions, e.g., preferably less than 10%, less than 5%, or less than 1%.

It will be evident that, while the exemplary compounds above include methoxysilane groups, other silane groups can be employed in place of the methoxysilane groups, producing similar compounds also of use in the present invention.

A surface to be modified can be contacted with a single surface modifying agent or with a mixture of agents. Thus, for example, to produce a non-reactive surface, the surface to be modified can be contacted with a compound comprising a PEG moiety coupled to two or more silane groups, where the compound does not include a reactive group (e.g., a methoxyPEG terminated branched silane such as that shown above). As another example, to produce a densely biotinylated surface, the surface to be modified can be contacted with a compound comprising a PEG moiety coupled to two or more silane groups and to one or more biotin groups (or, similarly, to essentially any other reactive group).

Alternatively, the surface can be modified with a combination of reagents instead of a single reagent. Thus, in one class of embodiments, the surface to be modified is contacted with a mixture of a first compound comprising a PEG moiety coupled to two or more silane groups and to a reactive group (or groups) and a second compound that does not include a reactive group (e.g., another PEG silane, e.g., another bi- or multipodal PEG-silane). By controlling the ratio of the first and second compounds (and optionally third, fourth, etc. compounds), the density of reactive groups on the modified surface can be readily controlled; see U.S. patent application Ser. No. 11/240,662. Surfaces with a low density of reactive groups, as described above, are optionally produced. For production of a surface having a low density of reactive groups, the first compound preferably includes a single PEG arm (as opposed to being a multi-armed PEG where incorporation of the reactive group in only a single one of the arms may not be readily achieved).

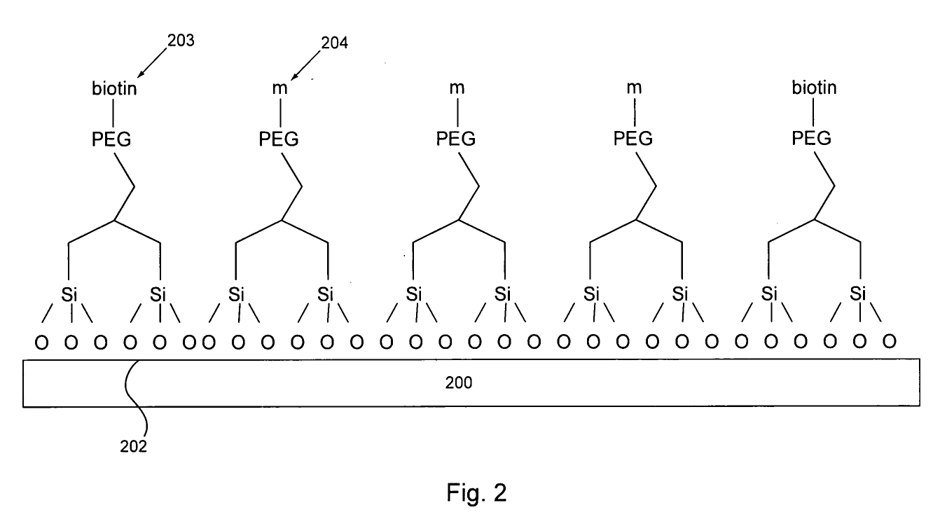

An exemplary embodiment is schematically illustrated in FIG. 2. Substrate 200 with glass or silica surface 202 is reacted with multipodal PEG-silanes 203 and 204. Silane 203 has a biotin reactive moiety, while silane 204 bears a non-reactive methoxyPEG group. The resulting modified surface thus has reactive groups (optionally at low density) disposed on an otherwise non-reactive surface.

The PEG-silanes described herein are optionally employed in orthogonal modification techniques such as those described in Ser. No. 11/731,748 and WO2007/123763, in which different materials in a hybrid substrate are selectively modified with different compounds. For example, in a ZMW that includes waveguide cores (apertures) disposed through a metal or metal oxide cladding layer to a transparent silicon or silicon oxide layer, the silica surfaces can be modified with a bi- or multipodal PEG-silane or mixture of silanes as described above (e.g., resulting in a low density of biotin or other moieties to which a polymerase can be attached), while the metal or metal oxide surfaces are passivated with phosphonates, polyelectrolyte-PEGs, polyelectrolyte multilayers, or the like.

Modification with Polymers Including Alkyl Phosphonates or Phosphates

There is a need for improved methods and compositions for surface modification of metal oxides in sensors, separation science, composites, and medicine. Phosphonic acids and phosphates have received considerable attention in the last ten years because the phosphate or phosphonic acid moiety binds strongly to metal oxides such as Ta₂O₅, TiO₂, Nb₂O₅, Al₂O₃, Fe₂O₃, ZrO₂, and SnO₂. Interestingly, these moieties do not bind strongly to SiO₂. This differentiation has been exploited for patterning surfaces with SiO₂ and other oxides.

It has been recognized that robust monolayers on surfaces exposed to water require the presence of an alkyl chain of certain length (typically, longer than six methyl groups) attached to the phosphate or phosphonic acid group. Use of alkyl-phosphonates and alkyl-phosphates (organophosphates and organophosphonates) for surface modification of oxides is taught, e.g., in U.S. patent application publication 2003/0186914 “Method for precipitating mono and multiple layers of organophosphoric and organophosphonic acids and the salts thereof in addition to use thereof” by Hofer et al. Typically, these materials consist of relatively low molecular weight linear molecules. However, Zoulalian et al. (2006) “Functionalization of titanium oxide surfaces by means of poly(alkyl-phosphonates)” J. Phys. Chem. B 110(51):25603-25605 teaches the synthesis and use of alkyl-phosphonates copolymerized with PEG44 (linear chain with 44 ethylene oxides) to impart robust biocompatible PEG-ylated surface characteristics to TiO₂. The presence of multiple alkyl-phosphor (phosphate or phosphonate) groups per polymer chain contribute to the stability (robustness) of the layer. However, Zoulalian et al. only considered a 1:1 ratio of PEG44 molecules to alkyl phosphor molecules per polymer. From basic chemical considerations, it is not possible to pack a dense monolayer of alkyl-phosphors if statistically there is one large PEG44 molecules per alkyl-phosphor.

This difficulty can be overcome by addition of another type of monomer to the polymeric structure to decrease the density of PEG chains relative to alkyl-phosphonate (or alkyl-phosphate) groups while preserving the biocompatibility of the coating.

Thus, for an exemplary copolymer formed from methacrylate-alkyl-phosphonate and mPEG-methacrylate monomers, the ratio of the methacrylate-alkyl-phosphonate monomer to the mPEG-methacrylate monomer is greater than 1:1, e.g., between 5:1 and 500:1. The copolymer also includes a methacrylic acid or a low molecular weight ethylene glycol methacrylate (e.g., EG3 methacrylate) monomer, for example, at a ratio between 5:4 and 500:499 methacrylate-alkyl-phosphonate monomer:(methacrylic acid or a low molecular weight ethylene glycol methacrylate monomer). In this way, alkyl chains are matched stoichiometrically to hydrophilic groups, and can form a dense monolayer on the substrate while allowing sufficient space to accommodate the large PEGylated groups as well.

It will be evident similar considerations apply to other types of monomers and copolymers. Polymerization is optionally performed prior to contact with the surface or in situ on the surface.

The resulting modified surfaces are optionally non-reactive. For example, methoxyPEG (or similar) containing copolymers can be used to passivate a metal oxide surface. Alternatively, the resulting modified surface can be reactive; thus, a fraction of the PEG repeats optionally include a reactive moiety. Exemplary reactive moieties are described above, and include ligands or recognition groups such as a biotin, SNAP-Tag™ or substrate therefore (Covalys Biosciences AG; the SNAP-Tag™ is a polypeptide based on mammalian 06-alkylguanine-DNA-alkyltransferase, and SNAP-tag substrates are derivates of benzyl purines and pyrimidines), NTA, RGDC peptides, and tethered nucleic acids. It will be evident that the density of reactive moieties on the surface is readily controlled, e.g., by controlling the degree of substitution of the PEG moieties.

The copolymers and/or methods of the invention can be employed in drug delivery systems, modification of medical implant devices, etc. In one aspect, the methods are employed to selectively coat (for passivation or to render specific activity) selected components of hybrid substrates whose components exhibit dissimilar surface characteristics, e.g., nanostructured substrates. For example, as noted above, ZMWs can be fabricated on silica substrates by opening nanosized holes in metallic films. Since phosphonates and phosphates do not bind strongly to SiO₂, the SiO₂ surface is not irreversibly modified by the copolymers described herein, while the metallic regions including the walls of the ZMW cores are modified. Alternatively, the phosphonate or phosphate copolymers can be employed to selectively modify the observation surface, rather than the walls, of a ZMW. In one example where the copolymers are employed to modify the observation surface, zirconium oxide (zirconia) is disposed on a fused silica wafer (by sputtering or other methods known in the art, such as sol-gel methods or thermal chemical vapor deposition) before the ZMW is manufactured by aluminum deposition. In the resulting device, the bottom of the ZMW (the observation region) has ZrO₂, a material that shows even higher affinity for phosphonates than does Al₂O₃ and that can thus be selectively modified by phosphonate compounds. The copolymers are optionally employed in orthogonal modification techniques such as those described in Ser. No. 11/731,748 and WO2007/123763, in which different materials in a hybrid substrate are selectively modified with different compounds.

It will be evident that, while the exemplary copolymers described herein employ PEG, non-PEGylated low protein adsorption moieties such as the antifouling peptoid moieties described in Dalsin and Messersmith (2005) “Bioinspired antifouling polymers” Materials Today 8(9):38-46, Statz et al. (2005) “New peptidomimetic polymers for antifouling surfaces” J. Am. Chem. Soc. 127:7972-7973, and U.S. patent application publication 2006/0241281 “Peptidomimetic polymers for antifouling surfaces” by Messersmith et al., or essentially any other anti-fouling moieties (for example, polyacrylamide, polypyrrolidone, polyvinyl alcohol, or dextrans), are applicable. (Use of one kind of reaction condition is desirable, to avoid problems with reaction quenching.) In addition, peptidomimetic polymers such as those described in U.S. patent application publication 2006/0241281 provide a useful polymer backbone.

As used herein, the term “phosphate group” refers to a group having the structure

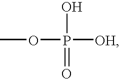

whether protonated, partially or completely deprotonated, and/or partially or completely neutralized (e.g., with K⁺, Na⁺, Li⁺, NH₄⁺, or the like). Similarly, the term “phosphonate group” or “phosphonic acid group” refers to a group having the structure

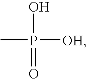

whether protonated, partially or completely deprotonated, and/or partially or completely neutralized (e.g., with K⁺, Na⁺, Li⁺, NH₄⁺, or the like).

The term “alkyl phosphate group” refers to a group having the structure

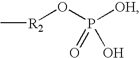

where R₂ is an alkyl group, a partially or totally fluorinated alkyl group, or an unsaturated hydrocarbon chain containing one or more double or triple bonds (again regardless of the protonation state of the phosphate group). The term “alkyl phosphonate group” refers to a group having the structure

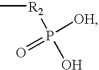

where R₂ is an alkyl group, a partially or totally fluorinated alkyl group, or an unsaturated hydrocarbon chain containing one or more double or triple bonds (again regardless of the protonation state of the phosphonate group).

Phosphonates and phosphates of interest in the invention generally include compounds of the form

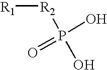

and

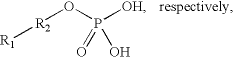

where (as evident from context) R₁ is part of a polymer, a reactive monomer (e.g. vinyl, acrylate, alkyne triple bond), or a capping reagent (e.g. thiol, carboxylate, Br, OH, amine, OH, epoxide), and where R₂ is an alkyl group, a partially or totally fluorinated alkyl group, or an unsaturated hydrocarbon chain containing one or more double or triple bonds (again regardless of the protonation state of the phosphate or phosphonate group). In embodiments in which R₂ is unsaturated, the double or triple bond(s) can serve as lateral crosslinking moieties to stabilize a self-assembled monolayer comprising the phosphonate or phosphate compound.

Linkage via Blocked Multivalent Binding Intermediates

Biotin binding molecules such as avidin and streptavidin are widely employed to immobilize biotinylated molecules of interest on surfaces bearing immobilized biotin. For example, a biotinylated polymerase or other molecule of interest can be immobilized via binding to avidin or streptavidin which is in turn bound to biotin on biotin-PEG-silane modified silica surfaces, as described herein and in U.S. patent application Ser. No. 11/240,662, 11/731,748 and WO2007/123763. However, avidin and streptavidin are tetrameric assemblies that usually contain four binding sites for biotin. The ability of avidin or streptavidin to bind up to four biotin moieties simultaneously can be a complicating factor in applications where a 1:1 ratio of molecule of interest to surface-immobilized biotin is desired in surface immobilization strategies. This issue can be addressed with dilution strategies as described in U.S. patent application Ser. No. 11/240,662. The methods of the invention provide additional approaches for ensuring 1:1 stoichiometric binding of biotinylated molecules of interest to surface-immobilized biotin-bearing ligands.

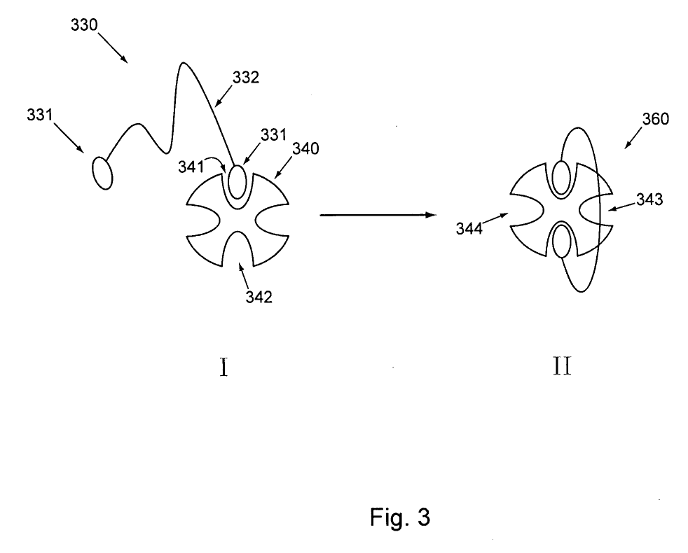

In one approach, two binding sites of the tetrameric biotin binding protein are specifically blocked through use of a bifunctional blocking reagent in solution. This approach is schematically illustrated in FIG. 3. Blocking reagent 330 includes two terminal biotin moieties 331 connected by linker 332. The linker is optionally between three and ten nm long, and is optionally a PEG moiety (e.g., blocking reagent 330 can be a biotin-PEG-biotin). The blocking reagent is contacted with tetrameric biotin binding protein 340 (e.g., avidin, streptavidin, or neutravidin) in a highly diluted solution, to statistically bind one copy of the blocking reagent per tetrameric protein (FIG. 3 Panel I). When a first copy of biotin on blocking reagent 330 binds to a binding site 341 on the tetramer, the second end of the blocking reagent will statistically wrap around and bind to a second binding site 342 on the same tetramer (Panel II). The resulting blocking reagent-tetramer complexes 360 can be concentrated and/or purified to isolate them from tetramers not bound to the blocking reagent and tetramers bound to two or more copies of the blocking reagent.

Blocking reagent-tetramer complex 360 represents a bifunctional ligand complex, which can be used with a biotinylated surface, preferably, a highly diluted biotinylated surface, so that statistically one site 343 per tetramer is available to bind the surface and the fourth site 344 per tetramer is available for biotin-mediated binding to a molecule of interest. (It will be evident that, while binding sites 341-344 are labeled for ease of discussion, they can in practice be equivalent.)

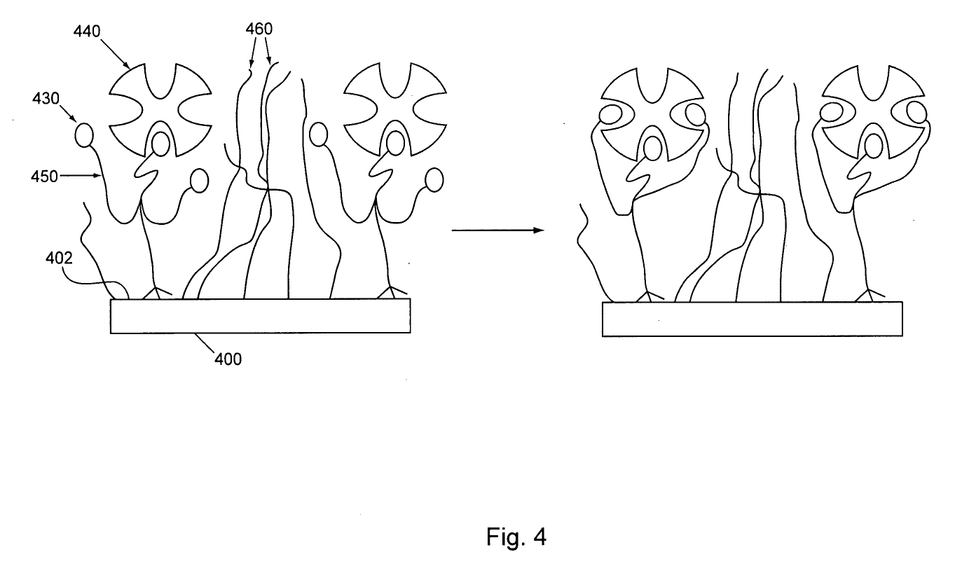

Another approach for specific immobilization of a single biotinylated molecule of interest per biotin-binding tetramer is schematically illustrated in FIG. 4. In this embodiment, surface 402 of substrate 400 is modified with a mixture of surface modifying agents 450 and 460, to produce a highly diluted surface of compound 450, which bears three biotin groups 430, in an otherwise non-reactive surface formed by compound 460 (which is not biotinylated). For example, compound 450 can be a tridentate biotin-PEG-silane (e.g., a trimethoxysilane), while compound 460 is a methoxyPEG-silane. The arms of the tri-functional compound 450 need to be long enough to wrap around the tetrameric biotin binding protein 440 (e.g., three to ten nm), but are preferably shorter than the average distance between biotinylated molecules 450 on the surface.

A dilute solution of biotin binding tetramer 440 is applied to the surface. When tetramer 440 is supplied at low concentration, it is statistically likely to have three binding sites blocked with locally dense biotins (from a single molecule of compound 450), leaving a single binding site open for binding to a biotinylated molecule of interest (e.g., polymerase). The exclusion of secondary tetramers (other tetramers binding to the same molecule 450) can be improved by making the “filler” compound 460 (e.g., the methoxyPEG-silane) with a longer PEG arm then the active biotinylated compound 450 (e.g., tri-biotin-PEG-silane).

As used herein, the terms avidin and streptavidin include wild type, mutant, glycosylated, deglycosylated (e.g., neutravidin), and/or other modified forms of these proteins, so long as they retain their characteristic ability to bind biotin.

Although described in terms of molecules of interest linked via biotin/avidin or streptavidin/biotin linkages to a derivatized surface, it will be apparent that the methods are applicable to essentially any other binding systems with multivalent binding intermediates, not just avidin and streptavidin; for example, multivalent antibodies, lectins, or the like.

Surfaces, substrates, and compositions produced by the methods of the invention are also features of the invention, as are devices and apparatus including such surfaces, substrates, and compositions.

### Exemplary Applications of Substrates and Surfaces

The surfaces and substrates of the invention have a variety of applications. For example, the selectively reactive surfaces of the invention have a variety of different applications where it may be desirable to isolate individual molecules or their reactions from each other. For example, bead substrates bearing single or few reactive molecules may be readily interrogated using FACS or other bead sorting methods, to ascertain a desired reactive group in, e.g., a combinatorial chemistry library, directed evolution library, or phage display library, or may be employed in bead-based assays as described in greater detail below. The surface modification techniques of the invention are applicable to such systems.

Single molecule analyses may be performed on a given enzyme system to monitor a single reaction and effectors of that reaction. Such analyses include enzyme assays that may be diagnostically or therapeutically important, such as kinase enzymes, phosphatase enzymes, protease enzymes, nuclease enzymes, polymerase enzymes, and the like.

Optionally, the surfaces are used to couple enzymes such as DNA polymerase enzymes at low densities in optically isolated/distinguishable locations on a substrate so as to analyze reactions such as sequencing reactions in real-time, and, e.g., to monitor and identify the sequence of the synthesis reactions as they occur. Examples of a particularly preferred application of the surfaces of the invention are described in published U.S. Patent Application No. 2003/0044781 and pending U.S. patent application Ser. No. 11/201,768, filed Aug. 11, 2005, which are incorporated herein by reference in their entirety for all purposes, and particularly, the application of such methods in zero mode waveguide structures as described in U.S. Pat. No. 6,917,726, previously incorporated herein by reference in its entirety for all purposes. In particular, sequencing data from the above described sequencing methods is more easily analyzed when data from individual reactions, i.e., individual polymerase enzymes, can be isolated from data from other enzymes. By providing such enzymes on a surface at a low density, one provides physical isolation, and thus the ability to optically isolate one enzyme from another. In its most preferred aspect, a single enzyme molecule would be provided upon the observation surface of each zero mode waveguide, to permit each waveguide to provide data for a reaction of a single enzyme molecule. Because it may be difficult to assure that every wave guide or other observation area possesses a single enzyme, a density is selected whereby many waveguides will include a single enzyme, while some will include 2 or 3 or more enzymes.

As will be appreciated, the highly defined surfaces of the invention may have application across a wide spectrum of applications, technologies and industries. For example, in other applications, the surfaces of the invention may be used in any of a variety of applications where it is desirable to precisely control the level of functionality of a surface to control the physical properties of such surfaces. For example, in a number of applications, precise control of ionic groups on a surface may provide precise control of the impact of such ionic groups on the surface's interaction with its environment. By way of example, in systems used for electrophoretic and/or electroosmotic transport of materials, e.g., in microfluidic conduits, e.g., channels, capillaries, etc., precise control of the zeta potential of the surface can have broad impacts upon the electroosmotic mobility of materials within such conduits, which can, in turn, impact the relative effectiveness of the system, e.g., in electrophoretic applications.

Further, in application of high surface area conduits, e.g., capillaries or channels, one may be desirous of maintaining a certain low level of functionality at a surface while preventing excessive interactions between materials and the surface. For example, in providing dynamic coatings for capillary electrophoresis a certain level of interaction between the coating material and the surface may be desired, while little or no interaction between analytes and the surface is desired.

In still other applications, the surfaces of the invention may be used to fine tune surface modifications on medical implants and grafts, to enhance biocompatibility of such devices, by more precisely controlling the level of surface modification thereon.

As noted previously, the substrates of the invention are, in preferred aspects, used in conjunction with optical detection systems to monitor particular reactions occurring on these low density surfaces. In particular, these systems typically employ fluorescence detection systems that include an excitation source, an optical train for directing excitation radiation toward the surface to be interrogated, and focusing emitted light from the substrate onto a detector. One example of such a system is set forth in U.S. patent application Ser. No. 11/201,768, filed Aug. 11, 2005, and incorporated herein by reference in its entirety for all purposes.

Bead-Based Single-Molecule Assays

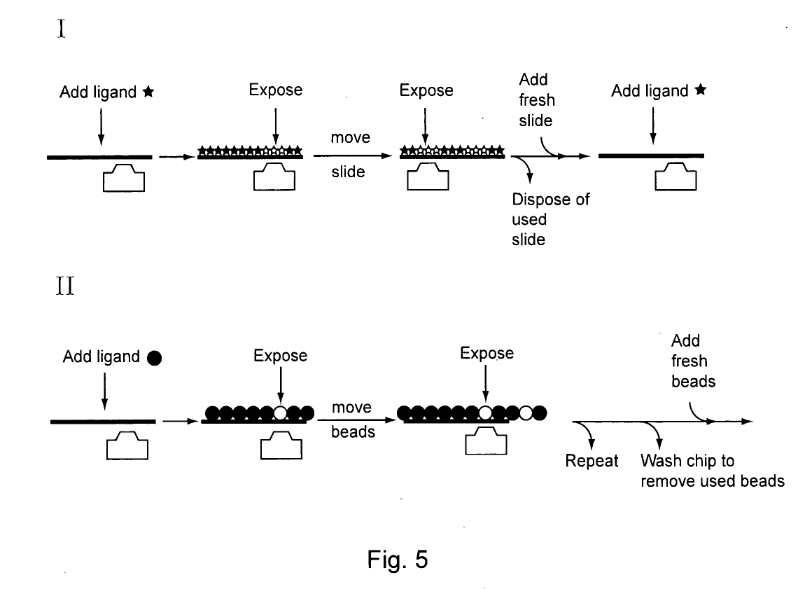

Most strategies for single-molecule assays or detection rely upon immobilizing one or more molecules of interest (e.g., an enzyme, a ligand, a reactant, etc.) to the surface of a microscope slide or coverslip before or during observation. This attachment tends to be semi-permanent or permanent, requiring manipulation, typically extensive manipulation, to return the surface to its original condition (if possible at all). Using this strategy, controlling the density of the molecule of interest on the surface often requires considerable and careful manipulation of the sample. In addition, viewing a ‘fresh’ molecule of interest or portion of the sample requires moving the slide or coverslip, illumination source, and/or detector relative to each other, as in the conventional microscope slide-based assay schematically illustrated in FIG. 5 Panel. I.

In one aspect of the invention, molecules of interest are immobilized on particles (e.g., beads) instead of on slides, coverslips, or similar planar substrates, using the methods described herein (or other surface modification techniques such as those described in U.S. patent application Ser. No. 11/240,662, 11/731,748 and WO2007/123763). The particle-bound molecules of interest are optionally employed in enzyme or binding assays (including, e.g., single-molecule reactions), high throughput screening, etc. The immobilized molecules on the particles can be viewed, for example, by conventional confocal microscopy, or preferably by total internal reflection microscopy (TIRF-M).

In contrast with the more typical immobilization of molecules on the surface of microscope slides, in this approach, the delivery, movement, exchange, and density of the molecule of interest are controlled by the preparation and movement of the beads rather than the microscope slide or objective. The microscope slide (or coverslip, microchannel, or other reaction region) remains unused and unaltered during the course of the reaction so it can be used multiple times without additional treatment. In addition, since the reaction takes place on a relatively mobile platform, the solution and beads can be moved to expose fresh molecule of interest without moving the stage or a detector, as schematically illustrated in FIG. 5 Panel II. The beads are optionally constructed from a magnetic material to facilitate temporary immobilization and movement of the beads. The relative size of the beads is optionally used to assist in controlling molecule density on the surface of the beads. The types of beads or other particles employed, the detection and illumination strategy, and the identity of the molecule immobilized on the beads are varied as desired.

In a preferred aspect, the molecule of interest is immobilized to the particles at low density such that when the beads are viewed, e.g., by TIRF, only a single molecule on average is detected in a single observation area, and the particles are employed in single-molecule analysis or detection. For example, in one embodiment, they are employed in single-molecule sequencing analysis. In this embodiment, the nucleic acid polymerase, the nucleic acid template, or the primer can be immobilized on the particles.

The particle-based assays of the invention have a number of advantageous features. For example, immobilizing the molecules of interest on particles provides two separate surfaces/media to work with (the surface of the particles and the surface of the support on which the assay is performed and/or analyzed) to increase flexibility of dealing with challenges related to immobilization and non-specific adhesion for the distinct components required for the reaction of interest. With reference to single molecule sequencing, for example, this provides potential for surface/immobilization specialization; for example, there may be different requirements to obtain specific binding to and/or rejection of the different components (proteins, nucleic acid, and nucleotide analogs) from the separate surfaces. As another advantage, immobilization of the polymerase or nucleic acid can be separated into a separate step or set of conditions, before the actual sequencing reaction is performed. Another advantage is the possibility of separate conditions or surfaces for rejection of non-specific analog sticking during the sequencing reaction; for example, the slide surface could be optimized to reject fluorophore (nucleotide analog) adhesion but have poor protein rejection—which would not matter since the polymerase is already immobilized on the bead in a separate, prior step. Another advantage is that unique modifications can be made to the surface to block or quench localized fluorescent interactions. For example, quenchers (e.g., Black Hole™ or other dark quenchers) can be applied to the surface of the glass slide on which the sequencing assay is performed to quench fluorophores that stick to that surface. Similarly, a metal/dielectric layer can be employed near or at the surface of the slide to quench fluorophores that are close to or stuck to the surface. As noted, bead size provides a simple way to control the density of the reactive complexes in the system, e.g., in a given observation volume or area. The surface of the particles may exhibit high specificity for certain reagents under specific conditions.

Yet another advantage to employing the molecule of interest immobilized on particles is the potential to separate optical requirements or properties for the different immobilization and reaction conditions. While optical properties of slides or other substrates in conventional assays are frequently important, the optical properties (e.g., dielectric, transmittivity, or autofluorescence) of the beads may not be important. In addition, the composition of the beads (surface and/or interior) can impact immobilization specificity of the reaction components, enabling use of alternative strategies for protein and nucleic acid immobilization and permitting alternative surface/composition chemistries to be optimized for biological function independent of optical aspects. This may be particularly useful for protein or nucleic acid binding specificity, which can be challenging due to the relative complexity of these macromolecules compared to small molecules.

Another advantageous feature is that the particles can be used to introduce or localize other reaction components that enhance various aspects of the assay. Again with reference to single-molecule sequencing, the particles can be used to co-localize (with the polymerase, template, primer, and/or complex thereof) components such as an oxygen-mitigation system on the surface of the beads, SAP for destruction of phosphates, DNA binding proteins for processivity enhancement of immobilized DNA polymerase, reagent(s) for phosphorolytic detection of cleavage products, quenchers to quench fluorophores that bind to other regions of the bead, and/or repair enzymes to fix nicked DNA or photodamage. Similarly, the particles can provide reagents for sample preparation, e.g., to generate DNA or RNA for sequencing.

Internal properties of the particles can also enhance such assays. For example, an oxygen-mitigation system (enzymatic or chemical) can be imbedded in the interior of the particles for localized oxygen removal, perhaps with increased efficiency if chemical systems is employed. The particles can be magnetic; this property is useful to move, remove, and/or immobilize the beads before, after, or during the reaction, respectively. Multiple FRET donors can be present in the core of the particles, to avoid problems with donor-bleaching (blinking) and to potentially reduce donor-related photodamage since the fluorophores would be isolated from the surface by the outer shell of the bead. Optical/dielectric properties of the particles can also be useful. For example, the particles can contain substance (e.g., metals) that enhance the local fields and preferentially alter the fluorescent properties of molecules that are localized to the surface of the beads (e.g., to decrease fluorescence lifetimes, increase brightness, enhance triplet state relaxation). Scattering can generate increased localized excitation in TIRF that could be wavelength specific. Opacity of the particles can increase signal-to-noise ratio by blocking signals from ‘behind’ the polymerase or other molecule of interest.

It will be evident that the mechanics of the instrument can be optimized for stability (e.g., focus, laser alignment, evanescent wave penetration depth, signal-to-noise ratio, etc.) since the relative position of the slide does not have to move to bring a new sample into the observation volume. The slide (or other support for the particles) can also be optimally positioned to maximize these different elements and to maximize repeatability (e.g., avoidance of defects, heterogeneity, thickness differences, autofluorescence, etc.)

While the foregoing invention has been described in some detail for purposes of clarity and understanding, it will be clear to one skilled in the art from a reading of this disclosure that various changes in form and detail can be made without departing from the true scope of the invention. For example, all the techniques and apparatus described above can be used in various combinations. All publications, patents, patent applications, and/or other documents cited in this application are incorporated by reference in their entirety for all purposes to the same extent as if each individual publication, patent, patent application, and/or other document were individually indicated to be incorporated by reference for all purposes.

What is claimed is:

1. A method of preparing a modified surface, the method comprising:
providing a surface to be modified;
copolymerizing at least three different monomers to form a polymer, wherein the at least three monomers comprise a first monomer comprising an alkyl phosphonate or alkyl phosphate group, a second monomer, and a third monomer, wherein the ratio of the first monomer to the third monomer is greater than 1:1; and
contacting the surface to be modified with the polymer to produce the modified surface having the polymer bound thereto.

2. The method of claim 1, wherein the surface to be modified comprises a metal oxide.

3. The method of claim 2, wherein the surface to be modified comprises Al₂O₃, Ta₂O₅, TiO₂, Nb₂O₅, Fe₂O₃, ZrO₂, or SnO₂.

4. The method of claim 1, wherein the ratio of the first monomer to the third monomer in the polymer is between 5:1 and 500:1.

5. The method of claim 1, wherein the ratio of the first monomer to the second monomer in the polymer is greater than 1:1.

6. The method of claim 5, wherein the ratio of the first monomer to the second monomer in the polymer is between 5:4 and 500:499.

7. The method of claim 1, wherein the ratio of the first to the second to the third monomer in the polymer is between 5:4:1 and 500:499:1.

8. The method of claim 1, wherein the ratio of the first monomer to the sum of the second and third monomers in the polymer is about 1:1.

9. The method of claim 1, wherein the first monomer is a methacrylate-alkyl-phosphonate.

10. The method of claim 1, wherein the third monomer comprises a polyethylene glycol.

11. The method of claim 1, wherein the third monomer is a polyethylene glycol methacrylate monomer or a polyethylene glycol methyl ether methacrylate monomer.

12. The method of claim 11, wherein the third monomer comprises a polyethylene glycol moiety with more than four ethylene glycol repeat units.

13. The method of claim 1, wherein the second monomer is methacrylic acid or a polyethylene glycol methacrylate monomer.

14. The method of claim 1, wherein the at least three monomers comprise four monomers, the four monomers comprising the first monomer, the second monomer, the third monomer, and a fourth monomer comprising a reactive moiety; wherein the first, second, and third monomers do not comprise the reactive moiety.

15. The method of claim 14, wherein the reactive moiety comprises a binding moiety.

16. The method of claim 1, wherein the surface comprises an observation area.

17. The method of claim 1, wherein the surface comprises an observation surface of an optical confinement.

18. A substrate comprising:
a metal oxide surface; and
a polymer layer disposed on the surface, which layer comprises a copolymer comprising at least a first monomer comprising an alkyl phosphonate or alkyl phosphate group, a second monomer, and a third monomer, wherein the ratio of the first monomer to the third monomer is greater than 1:1.

19. The substrate of claim 18, wherein the copolymer comprises the first monomer, the second monomer, the third monomer, and a fourth monomer comprising a reactive moiety;
wherein the first, second, and third monomers do not comprise the reactive moiety.

20. A zero mode waveguide array comprising the substrate of claim 18.

21. A method of preparing a modified surface, the method comprising:
providing a surface to be modified;
providing a first surface modifying agent, which first surface modifying agent comprises a polyethylene glycol moiety coupled to two or more silane groups; and
contacting the surface to be modified with the first surface modifying agent, to produce the modified surface having the first surface modifying agent coupled thereto.

22. The method of claim 21, wherein the first surface modifying agent comprises a reactive moiety coupled to the polyethylene glycol moiety.

23. The method of claim 22, wherein the reactive moiety comprises a binding moiety.

24. The method of claim 23, wherein the binding moiety comprises a specific binding moiety.

25. The method of claim 23, wherein the binding moiety is selected from the group of consisting of an antigen, an antibody, an binding fragment of an antibody, a polynucleotide, a binding peptide, biotin, avidin and streptavidin.

26. The method of claim 22, wherein the reactive moiety comprises a catalytic moiety.

27. The method of claim 26, wherein the catalytic moiety comprises an enzyme.

28. The method of claim 27, wherein the enzyme is selected from a nucleic acid polymerase, a ligase, a nuclease, a protease, a kinase and a phosphatase.

29. The method of claim 27, wherein the enzyme comprises a DNA polymerase.

30. The method of claim 22, comprising:
providing a second surface modifying agent, which second surface modifying agent comprises a polyethylene glycol moiety coupled to two or more silane groups, and which second surface modifying agent does not comprise the reactive moiety; and
forming a mixture of the first and second surface modifying agents;
wherein contacting the surface to be modified with the first surface modifying agent comprises contacting the surface with the mixture to produce the modified surface having the first and second modifying agents coupled thereto.

31. The method of claim 21, wherein the first surface modifying agent preferentially couples to the surface rather than undergoing an intramolecular reaction.

32. The method of claim 21, wherein the silane groups are trimethoxysilane groups.

33. The method of claim 21, wherein the surface comprises an observation area.

34. The method of claim 33, wherein the observation area comprises the observation surface of a zero mode waveguide.

35. The method of claim 21, wherein the surface comprises an observation surface of an optical confinement.

36. The method of claim 21, wherein the surface comprises silica.

37. The method of claim 21, wherein the surface comprises a material selected from glass, quartz, fused silica, and silicon.

38. A substrate comprising: a surface to which is coupled a first surface modifying agent, which first surface modifying agent comprises a polyethylene glycol moiety coupled to two or more silane groups.

39. The substrate of claim 38, wherein the first surface modifying agent comprises a reactive moiety coupled to the polyethylene glycol moiety.

40. The substrate of claim 39, wherein a second surface modifying agent is coupled to the surface, which second surface modifying agent comprises a polyethylene glycol moiety coupled to two or more silane groups, and which second surface modifying agent does not comprise the reactive moiety.

41. A zero mode waveguide array comprising the substrate of claim 38.

42. A method of immobilizing a desired molecule on a surface, the method comprising:
providing the surface on which the molecule is to be immobilized;
coupling a first copy of a first binding moiety to the surface;
providing a multivalent binding intermediate which has three or more binding sites for the first binding moiety;
binding the multivalent binding intermediate to the first copy of the first binding moiety coupled to the surface, thereby coupling the multivalent binding intermediate to the surface;
blocking one or more of the binding sites on the multivalent binding intermediate to produce a blocked multivalent binding intermediate;
providing a desired molecule coupled to a second copy of the first binding moiety; and
binding the second copy of the first binding moiety to the blocked multivalent binding intermediate, thereby coupling the desired molecule to the multivalent binding intermediate.

43. The method of claim 42, wherein the first binding moiety is biotin and the multivalent binding intermediate comprises an avidin or streptavidin.

44. The method of claim 42, wherein the multivalent binding intermediate has four binding sites for the first binding moiety; and wherein blocking one or more of the binding sites on the multivalent binding intermediate comprises blocking two of the binding sites.

45. The method of claim 44, wherein blocking two of the binding sites on the multivalent binding intermediate comprises:
providing a blocking reagent which comprises two copies of the first binding moiety coupled by a linker;
contacting the blocking reagent with the multivalent binding intermediate and permitting the two copies of the first binding moiety to occupy two of the binding sites on the multivalent binding intermediate, to produce the blocked multivalent binding intermediate; and
optionally isolating the blocked multivalent binding intermediate.

46. The method of claim 44, wherein coupling a first copy of the first binding moiety to the surface comprises coupling a first surface modifying agent to the surface, which first surface modifying agent comprises three copies of the first binding moiety; and wherein binding the multivalent binding intermediate to the first copy of the first binding moiety and blocking two of the binding sites on the multivalent binding intermediate comprises contacting the multivalent binding intermediate and the surface-coupled first surface modifying agent and permitting the three copies of the first binding moiety to occupy three of the binding sites on the multivalent binding intermediate, to provide the blocked multivalent binding intermediate.

47. The method of claim 46, wherein the first surface modifying agent is a biotin-PEG-silane comprising three biotin moieties.

48. The method of claim 46, comprising coupling a second surface modifying agent to the surface, which second surface modifying agent does not comprise the first binding moiety, and which second surface modifying agent is present in excess of the first surface modifying agent.

49. A method of performing a reaction involving a molecule of interest, the method comprising:
a) providing particles having the molecule of interest coupled to their surface;
b) positioning a first subset of the particles in an observation area;
c) performing the reaction;
d) removing the first subset of particles from the observation area; and
e) repeating steps b-d with a second subset of the particles.

50. The method of claim 49, wherein the molecule of interest is coupled to the surface of the particles at a density selected so that from 1 to 3 molecules of interest are within the observation area when the first subset of particles is positioned in the observation area.

51. The method of claim 49, wherein the molecule of interest is an enzyme.

52. The method of claim 51, wherein the molecule of interest is a DNA polymerase.

53. The method of claim 49, wherein the molecule of interest is a nucleic acid.

54. The method of claim 53, wherein the nucleic acid is configured to serve as a template or primer in a nucleic acid sequencing reaction.

55. The method of claim 49, comprising monitoring the reaction by confocal microscopy or total-internal reflection microscopy.

56. An optically distinguishable single molecule reaction comprising a nucleic acid template or primer bound to a particle.

57. The single molecule reaction of claim 56, further comprising an enzyme bound to a particle.

58. The single molecule reaction of claim 56, wherein the particle is a bead or a nanoparticle.

59. The single molecule reaction of claim 56, wherein the particle comprises a metal, a magnetic material, a quencher, a fluorescent donor, a plurality of fluorescent donors, or a metal/dielectric layer.

60. The single molecule reaction of claim 56, wherein the reaction is a DNA sequencing reaction.

61. A single molecule reaction comprising a first reactant or reagent bound to a first particle, and a second reactant or reagent bound to a second particle, wherein the first reactant or reagent and the second reactant or reagent are different, and wherein the first and second particles are different.

62. The single molecule reaction of claim 61, wherein the first and second particles each comprise a different surface modification.
